SEQUENCE LISTING (56 sequences)

SEQ ID NO: 1 - DNA, 1293 bp, Arabidopsis thaliana
  G979
atgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactagacatagatggactgggagattcgaggctcatctttgggacaaaagctcttggaattcgattcagaacaagaaaggcaaacaagtttatctgggagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagagcccaccctcttcttcttcaccattgtcttgcttatctactgactctgcttcatcaacaacaacaacaacaacctcggtttcttgtaactatttggtctga

SEQ ID NO: 2 - protein, 430 aa, Arabidopsis thaliana
  G979 polypeptide, AP2 domains 64-133,166-227, linker domain 134-165
MKKRLTTSTCSSSPSSSVSSSTTTSSPIQSEAPRPKRAKRAKKSSPSGDKSHNPTSPASTRRSSIYRGVTRHRWTGRFEAHLWDKSSWNSIQNKKGKQVYLGAYDSEEAAAHTYDLAALKYWGPDTILNFPAETYTKELEEMQRVTKEEYLASLRRQSSGFSRGVSKYRGVARHHHNGRWEARIGRVFGNKYLYLGTYNTQEEAAAAYDMAAIEYRGANAVTNFDISNYIDRLKKKGVFPFPVNQANHQEGILVEAKQEVETREAKEEPREEVKQQYVEEPPQEEEEKEEEKAEQQEAEIVGYSEEAAVVNCCIDSSTIMEMDRCGDNNELAWNFCMMDTGFSPFLTDQNLANENPIEYPELFNELAFEDNIDFMFDDGKHECLNLENLDCCVVGRESPPSSSSPLSCLSTDSASSTTTTTTSVSCNYLV

SEQ ID NO: 3 - DNA, 1188 bp, Zea mays
  G5297
atggagagatctcaacggcagtctcctccgccaccgtcgccgtcctcctcctcgtcctccgtctccgcggacaccgtcctcgtccctcccggaaagaggcggagggcggcgacggccaaggccggcgccgagcctaataagaggatccgcaaggaccccgccgccgccgccgcggggaagaggagctccgtctacaggggagtcaccaggcacaggtggacgggcaggttcgaggcgcatctctgggacaagcactgcctcgccgcgctccacaacaagaagaaaggcaggcaagtctacctgggggcgtatgacagcgaggaggcagctgctcgtgcctatgacctcgcagctctcaagtactggggtcctgagactctgctcaacttccctgtggaggattactccagcgagatgccggagatggaggccgtgtcccgggaggagtacctggcctccctccgccgcaggagcagcggcttctccaggggcgtctccaagtacagaggcgtcgccaggcatcaccacaacgggaggtgggaggcacggattgggcgagtctttgggaacaagtacctctacttgggaacatttgacactcaagaagaggcagccaaggcctatgaccttgcggccattgaataccgtggcgtcaatgctgtaaccaacttcgacatcagctgctacctggaccacccgctgttcctggcacagctccaacaggagccacaggtggtgccggcactcaaccaagaacctcaacctgatcagagcgaaaccggaactacagagcaagagccggagtcaagcgaagccaagacaccggatggcagtgcagaacccgatgagaacgcggtgcctgacgacaccgcggagcccctcaccacagtcgacgacagcatcgaagagggcttgtggagcccttgcatggattacgagctagacaccatgtcgagaccaaactttggcagctcaatcaatctgagcgagtggttcgctgacgcagacttcgactgcaacatcggatgcctgttcgatgggtgttctgcggctgacgaaggaagcaaggatggtgtaggtctggcagatttcagtctgtttgaggcaggtgatgtccagctgaaggatgttctttcggatatggaagaggggatacaacctccagcgatgatcagtgtgtgcaactaa

SEQ ID NO: 4 - protein, 395 aa, Zea mays
  G5297 polypeptide, AP2 domains 63-133, 166-227, linker domain 134-165
MERSQRQSPPPPSPSSSSSSVSADTVLVPPGKRRRAATAKAGAEPNKRIRKDPAAAAAGKRSSVYRGVTRHRWTGRFEAHLWDKHCLAALHNKKKGRQVYLGAYDSEEAAARAYDLAALKYWGPETLLNFPVEDYSSEMPEMEAVSREEYLASLRRRSSGFSRGVSKYRGVARHHHNGRWEARIGRVFGNKYLYLGTFDTQEEAAKAYDLAAIEYRGVNAVTNFDISCYLDHPLFLAQLQQEPQVVPALNQEPQPDQSETGTTEQEPESSEAKTPDGSAEPDENAVPDDTAEPLTTVDDSIEEGLWSPCMDYELDTMSRPNFGSSINLSEWFADADFDCNIGCLFDGCSAADEGSKDGVGLADFSLFEAGDVQLKDVLSDMEEGIQPPAMISVCN

SEQ ID NO: 5 - DNA, 1197 bp, Zea mays
  G5286
atggagagatctcaacggcagtctcctccgccaccgtcgccgtcgtcctcctcgtcctgcgtctccgcggacaccgtcctcgtccctccgggaaagaggcggcggagggcggcgacggccaaggccggcgccgagcctaataagagggcccgcaaggacccctctgatcctcctcccgccgccggggagaggagctccgtctacaggggagtcaccaggcacaggtggacgggcaggttcgaggcgcatctctgggacaagcactgcctcgccgcgctccacaacaagaagaaaggcaggcaagtctacctgggggcgtatgacagcgaggaggcagctgctcgtgcctatgacctcgcagctctcaagtactggggtcctgagactctgctcaacttccctgtggaggattactccagcgagatgccggagatggaggccgtgtcccgggaggagtacctggcctccctccgccgcaggagcagcggcttctccaggggcgtctccaagtacagaggcgtcgccaggcatcaccacaacgggaggtgggaggcacggattgggcgagtctttgggaacaagtacctctacttgggaacatttgacactcaagaagaggcagccaaggcctatgaccttgcggccattgaataccgtggcgtcaatgctgtaaccaacttcgacatcagctgctacctggaccacccgctgttcctggcacagctccaacaggagccacaggtggtgccggcactcaaccaagaacctcaacctgatcagagcgaaaccggaactacagagcaagagccggagtcaagcgaagccaagacaccggatggcagtgcagaacccgatgagaacgcggtgcctgacgacaccgcggagcccctcaccacagtcgacgacagcatcgaagagggcttgtggagcccttgcatggattacgagctagacaccatgtcgagaccaaactttggcagctcaatcaatctgagcgagtggttcgctgacgcagacttcgactgcaacatcggatgcctgttcgatgggtgttctgcggctgacgaaggaagcaaggatggtgtaggtctggcagatttcagtctgtttgaggcaggtgatgtccagctgaaggatgttctttcggatatggaagaggggatacaacctccagcgatgatcagtgtgtgcaactaa

SEQ ID NO: 6 - protein, 398 aa, Zea mays
  G5286 polypeptide, AP2 domains 66-136, 169-230, linker domain 137-168
MERSQRQSPPPPSPSSSSSCVSADTVLVPPGKRRRRAATAKAGAEPNKRARKDPSDPPPAAGERSSVYRGVTRHRWTGRFEAHLWDKHCLAALHNKKKGRQVYLGAYDSEEAAARAYDLAALKYWGPETLLNFPVEDYSSEMPEMEAVSREEYLASLRRRSSGFSRGVSKYRGVARHHHNGRWEARIGRVFGNKYLYLGTFDTQEEAAKAYDLAAIEYRGVNAVTNFDISCYLDHPLFLAQLQQEPQVVPALNQEPQPDQSETGTTEQEPESSEAKTPDGSAEPDENAVPDDTAEPLTTVDDSIEEGLWSPCMDYELDTMSRPNFGSSINLSEWFADADFDCNIGCLFDGCSAADEGSKDGVGLADFSLFEAGDVQLKDVLSDMEEGIQPPAMISVCN

SEQ ID NO: 7 - DNA, 1341 bp, Oryza sativa
  G5285
atggcgaagagatcgtctcctgatcccgcatcatcttctccatctgcatcatcctcgccgtcgtctccttcctcctcttcctccgaggattcctcttcgcccatgtcgatgccctgcaagaggagggcgaggccgaggacggacaagagcaccggcaaggccaagaggcccaagaaggagagcaaggaggtggttgatccttcttccaatggcggcggcggcggcaagaggagttctatctacaggggagtcaccaggcatcggtggactggcagatttgaggcccatctgtgggacaagaattgctccacttcacttcagaacaagaagaaagggaggcaagtctatttgggggcttatgatagtgaagaggcagctgctcgtgcatatgaccttgcagctcttaagtactggggtcctgagacagtgctcaatttcccactggaggaatatgagaaggagaggtcggagatggagggtgtgtcgagggaggagtacctggcctccctccgccgccggagcagcggtttctccaggggtgtctccaagtacagaggcgttgccaggcatcaccacaatgggcggtgggaggcacggatagggcgggtcctggggaacaagtacctctacctgggtactttcgatactcaagaggaggcagccaaggcctatgatcttgctgcaattgaataccgaggtgccaatgcggtaaccaacttcgacatcagctgctacctggaccagccacagttactggcacagctgcaacaggaaccacagttactggcacaactgcaacaagagctacaggtggtgccagcattacatgaagagcctcaagatgatgaccgaagtgagaatgcagtccaagagctcagttccagtgaagcaaatacatcaagtgacaacaatgagccacttgcagccgatgacagcgctgaatgcatgaatgaaccccttccaattgttgatggcattgaagaaagcctctggagcccttgcttggattatgaattggatacaatgcctggggcttacttcagcaactcgatgaatttcagtgaatggttcaatgatgaggctttcgaaggcggcatggagtacctatttgaagggtgctccagtataactgaaggcggcaacagcatggataactcaggtgtgacagaatacaatttgtttgaggaatgcaatatgttggagaaggacatttcagattttttagacaaggacatttcagattttttagataaggacatttcaatttcagatagggagcgaatatctcctcaagcaaacaatatctcctgccctcaaaaaatgatcagtgtgtgcaactga

SEQ ID NO: 8 - protein, 446 aa, Oryza sativa
  G5285 polypeptide, AP2 domains 79-149, 182-243, linker domain 150-181
MAKRSSPDPASSSPSASSSPSSPSSSSSEDSSSPMSMPCKRRARPRTDKSTGKAKRPKKESKEVVDPSSNGGGGGKRSSIYRGVTRHRWTGRFEAHLWDKNCSTSLQNKKKGRQVYLGAYDSEEAAARAYDLAALKYWGPETVLNFPLEEYEKERSEMEGVSREEYLASLRRRSSGFSRGVSKYRGVARHHHNGRWEARIGRVLGNKYLYLGTFDTQEEAAKAYDLAAIEYRGANAVTNFDISCYLDQPQLLAQLQQEPQLLAQLQQELQVVPALHEEPQDDDRSENAVQELSSSEANTSSDNNEPLAADDSAECMNEPLPIVDGIEESLWSPCLDYELDTMPGAYFSNSMNFSEWFNDEAFEGGMEYLFEGCSSITEGGNSMDNSGVTEYNLFEECNMLEKDISDFLDKDISDFLDKDISISDRERISPQANNISCPQKMISVCN

SEQ ID NO: 9 - DNA, 1242 bp, Brassica napus
  G5289
atgaagagacccttaaccacttctccttcttcctcctcttctacttcttcttcggcctgtatacttccgactcaatcagagactccaaggcccaaacgagccaaaagggctaagaaatcttctctgcgttctgatgttaaaccacagaatcccaccagtcctgcctccaccagacgcagctctatctacagaggagtcactagacatagatggacagggagatacgaagctcatctatgggacaaaagctcgtggaattcgattcagaacaagaaaggcaaacaagtttatctgggagcatatgacagcgaggaagcagcagcacatacgtacgatctagctgctctcaagtactggggtcccaacaccatcttgaactttccggttgagacgtacacaaaggagctggaggagatgcagagatgtacaaaggaagagtatttggcttctctccgccgccagagcagtggtttctctagaggcgtctctaaatatcgcggcgtcgccaggcatcaccataatggaagatgggaagctcggattggaagggtgtttggaaacaagtacttgtacctcggcacctataatacgcaggaggaagctgcagctgcatatgacatggcggctatagagtacagaggtgcaaacgcagtgaccaacttcgacattggtaactacatcgaccggttaaagaaaaaaggtgtcttcccgttccccgtgagccaagctaatcatcaagaagctgttcttgctgaaaccaaacaagaagtggaagctaaagaagagcctacagaagaagtgaagcagtgtgtcgaaaaagaagaagctaaagaagagaagactgagaaaaaacaacaacaagaagtggaggaggcggtgatcacttgctgcattgattcttcagagagcaatgagctggcttgggacttctgtatgatggattcagggtttgctccgtttttgactgattcaaatctctcgagtgagaatcccattgagtatcctgagcttttcaatgagatgggttttgaggataacattgacttcatgttcgaggaagggaagcaagactgcttgagcttggagaatcttgattgttgcgatggtgttgttgtggtgggaagagagagcccaacttcattgtcgtcttctccgttgtcctgcttgtctactgactctgcttcatcaacaacaacaacagcaacaacagtaacctctgtttcttggaactattctgtctga

SEQ ID NO: 10 - protein, 413 aa, Oryza sativa
  G5289 polypeptide, AP2 domains 61-130, 163-224, linker domain 131-162
MKRPLTTSPSSSSSTSSSACILPTQSETPRPKRAKRAKKSSLRSDVKPQNPTSPASTRRSSIYRGVTRHRWTGRYEAHLWDKSSWNSIQNKKGKQVYLGAYDSEEAAAHTYDLAALKYWGPNTILNFPVETYTKELEEMQRCTKEEYLASLRRQSSGFSRGVSKYRGVARHHHNGRWEARIGRVFGNKYLYLGTYNTQEEAAAAYDMAAIEYRGANAVTNFDIGNYIDRLKKKGVFPFPVSQANHQEAVLAETKQEVEAKEEPTEEVKQCVEKEEAKEEKTEKKQQQEVEEAVITCCIDSSESNELAWDFCMMDSGFAPFLTDSNLSSENPIEYPELFNEMGFEDNIDFMFEEGKQDCLSLENLDCCDGVVVVGRESPTSLSSSPLSCLSTDSASSTTTTATTVTSVSWNYSV

SEQ ID NO: 11 - DNA, 1065 bp, Arabidopsis thaliana
  G2131
gtctctcattttcataattccattttcaggattgtctctcaatcttttattcttctcattcaccggtaatggcaaaagtctctgggaggagcaagaaaacaatcgttgacgatgaaatcagcgataaaacagcgtctgcgtctgagtctgcgtccattgccttaacatccaaacgcaaacgtaagtcgccgcctcgaaacgctcctcttcaacgcagctccccttacagaggcgtcacaaggcatagatggactgggagatacgaagcgcatttgtgggataagaacagctggaacgatacacagaccaagaaaggacgtcaagtttatctaggggcttacgacgaagaagaagcagcagcacgtgcctacgacttagcagcattgaagtactggggacgagacacactcttgaacttccctttgccgagttatgacgaagacgtcaaagaaatggaaggccaatccaaggaagagtatattggatcattgagaagaaaaagtagtggattttctcgcggtgtatcaaaatacagaggcgttgcaaggcatcaccataatgggagatgggaagctagaattggaagggtgtttggtaataaatatctatatcttggaacatacgccacgcaagaagaagcagcaatcgcctacgacatcgcggcaatagagtaccgtggacttaacgccgttaccaatttcgacgtcagccgttatctaaaccctaacgccgccgcggataaagccgattccgattctaagcccattcgaagccctagtcgcgagcccgaatcgtcggatgataacaaatctccgaaatcagaggaagtaatcgaaccatctacatcgccggaagtgattccaactcgccggagcttccccgacgatatccagacgtattttgggtgtcaagattccggcaagttagcgactgaggaagacgtaatattcgattgtttcaattcttatataaatcctggcttctataacgagtttgattatggaccttaatcgtattttctacaagttttgttttgattatctacacaatacatcaatatattct

SEQ ID NO: 12 - protein, 313 aa, Arabidopsis thaliana
  G2131 polypeptide, AP2 domains 51-120,153-214, linker domain 121-152
MAKVSGRSKKTIVDDEISDKTASASESASIALTSKRKRKSPPRNAPLQRSSPYRGVTRHRWTGRYEAHLWDKNSWNDTQTKKGRQVYLGAYDEEEAAARAYDLAALKYWGRDTLLNFPLPSYDEDVKEMEGQSKEEYIGSLRRKSSGFSRGVSKYRGVARHHHNGRWEARIGRVFGNKYLYLGTYATQEEAAIAYDIAAIEYRGLNAVTNFDVSRYLNPNAAADKADSDSKPIRSPSREPESSDDNKSPKSEEVIEPSTSPEVIPTRRSFPDDIQTYFGCQDSGKLATEEDVIFDCFNSYINPGFYNEFDYGP

SEQ ID NO: 13 - DNA, 1126 bp, Arabidopsis thaliana
  G2106
cctcttcttttatgttcatcgccgtcgaagtttctccggtaatggaagacatcacacggcagagcaaaaaaacttcggttgagaatgaaaccggcgatgatcagtcagcaacatcagtagtccttaaagctaaacgcaaacgccgatcgcaaccacgagacgctccaccccaacgtagctccgtccatagaggcgtcacaaggcatcgatggactggaaggtacgaagcacatttgtgggataagaatagttggaacgaaactcagaccaagaaaggaagacaagtatatttaggggcatatgacgaggaagatgcagcagcacgtgcctacgacttagcagcattgaaatattggggacgagacaccatcttgaacttccctgtaaattttctcggaatcctattgtgtaattatgaagaagacatcaaagaaatggaaagccagtcaaaggaagagtatattggatctttgagaagaaaaagtagtgggttttcacgaggtgtatcaaaatacagaggcgttgcaaagcatcaccacaatgggagatgggaagctcgaatcggaagagtgtttggcaataaatatttataccttggaacttacgcgacgcaagaagaagcagctatagcgtacgatatcgcagctatcgagtaccgtggactcaacgccgttactaacttcgacatcagccgttatctgaaactcccggtgccggagaaccctatcgataccgcgaataatctcctcgagagtccgcattctgatcttagcccatttataaaacctaaccacgagtctgacttatcacagagtcaatcttcgtcagaggacaacgatgatcggaaaacaaagctcttgaagtcgtcacctttagtggcagaggaggtaatcggaccatcgacgccacctgagattgctccgcctcgtcggagcttcccggaagatatccagacgtatttcgggtgtcaaaactccggcaagttaacggcggaggaagatgatgttatcttcggtgatttagattctttccttacgcctgatttctacagcgagttaaatgattgctaaagtgttgttcttctgataagttttgttttttagttgttcagaatctcggttgtgaa

SEQ ID NO: 14 - protein, 352 aa, Arabidopsis thaliana
  G2106 polypeptide, AP2 domains 57-126,166-227, linker domain 134-165
MFIAVEVSPVMEDITRQSKKTSVENETGDDQSATSVVLKAKRKRRSQPRDAPPQRSSVHRGVTRHRWTGRYEAHLWDKNSWNETQTKKGRQVYLGAYDEEDAAARAYDLAALKYWGRDTILNFPVNFLGILLCNYEEDIKEMESQSKEEYIGSLRRKSSGFSRGVSKYRGVAKHHHNGRWEARIGRVFGNKYLYLGTYATQEEAAIAYDIAAIEYRGLNAVTNFDISRYLKLPVPENPIDTANNLLESPHSDLSPFIKPNHESDLSQSQSSSEDNDDRKTKLLKSSPLVAEEVIGPSTPPEIAPPRRSFPEDIQTYFGCQNSGKLTAEEDDVIFGDLDSFLTPDFYSELNDC

SEQ ID NO: 15 - DNA, 1866 bp, Arabidopsis thaliana
  G5288
aaggctagccgcctcactccctctctctgtttcctcttcttcttcttcctcccgccggtcagctcagctcgcctcgtctcctccattttggcgacgcgagcgagcatattaaagctgtgccggcggctgcaactttgccgccatttatttagctccggctcttttaaaagctctcttctcctgctgccatcttcttctggttggcaccaccattccatatataccatctccctcctcctcccgcgctcgctcttcgccaatggccaagcgacgcagcaacggcgagaccgccgccgcgagcagcgacgactctagctccggcgtctgcggcggcggcggcggcggtgaggttgagccgaggcggcggcagaagcggccgcggaggagcgccccgcgggattgcccctcccagcgcagctccgcgttccgcggcgtcacacggcaccggtggacggggcggttcgaggcgcatctctgggacaagaacacctggaacgagtcgcagagcaagaagggcagacaagtttacctcggggcttacgacggcgaggaagcggcggcgcgcgcctacgacctcgccgcattgaagtactggggccacgacaccgtcctcaacttccctctgtcaacatatgacgaggaattgaaggaaatggaggggcagtccagggaagagtacatcggatcgctccggaggaagagcagtggcttctcaagaggggtgtccaagtacagaggagttgcaaggcatcatcacaacggcaaatgggaggctcggattgggcgtgtgttcggcaacaaatacctctacctaggtacttatgcaacacaagaggaggcggccgtggcgtacgacatcgcggcgatcgagcaccgcggcctcaacgccgtcaccaacttcgacatcaatctctacatcaggtggtaccacggctcttgccgctccagcagcgccgccgccgccaccaccatcgaagacgatgatttcgccgaagccatcgccgccgcgttgcaaggcgtcgacgagcagccgtcgtcgtcgccggcgacgacgcgccagctgcaaaccgcggacgacgacgacgacgacctcgtggcgcagctcccgccccagctgaggccgctggctcgcgcggcgtccacctccccgatcggactgctgctgcggtcgcccaagttcaaggagatcatcgagcaggcggcggccgcggcggcgtcgtcctctggtagcagcagtagcagcagcacagactcaccttcttcttcgtcgtcgtcatcgctgtcgccgtcgccattgccatcgccgccgccgcagcagcagccaaccgtaccgaaggacgaccagtacaacgtcgacatgtcgtcggtggcggcggcgaggtgcagcttcccggacgacgtgcagacgtacttcgggctggacgacgacggcttcgggtacccggaggtggacacgttcttgttcggggatttgggcgcgtacgcggcgcccatgtttcagttcgagctcgacgtctgaactctcaactccgaccagggtgtttcgggaggcccacaatcccagcctgttcccgtagatgggctggaaagatcgaatcaaatttgggcctattcaggggatgggctgggagaattgatatgggccggcagggatggccgaaagggaaggcctcccaagttttggctgtcaagaagctagaactgagttctctctcaaaagagagagagagagagaagctagaactgaggattagttgcttactcgcaaatactagtagtttggaggaagagtaaaatattggtttatttgttgcccatctctgcgaaggggaatttaccgtaataaagagtacatattgccgtttt

SEQ ID NO: 16 - protein, 419 aa, Arabidopsis thaliana
  G5288 polypeptide, AP2 domains 54-123,156-217, linker domain 124-155
MAKRRSNGETAAASSDDSSSGVCGGGGGGEVEPRRRQKRPRRSAPRDCPSQRSSAFRGVTRHRWTGRFEAHLWDKNTWNESQSKKGRQVYLGAYDGEEAAARAYDLAALKYWGHDTVLNFPLSTYDEELKEMEGQSREEYIGSLRRKSSGFSRGVSKYRGVARHHHNGKWEARIGRVFGNKYLYLGTYATQEEAAVAYDIAAIEHRGLNAVTNFDINLYIRWYHGSCRSSSAAAATTIEDDDFAEAIAAALQGVDEQPSSSPATTRQLQTADDDDDDLVAQLPPQLRPLARAASTSPIGLLLRSPKFKEIIEQAAAAAASSSGSSSSSSTDSPSSSSSSSLSPSPLPSPPPQQQPTVPKDDQYNVDMSSVAAARCSFPDDVQTYFGLDDDGFGYPEVDTFLFGDLGAYAAPMFQFELDV

SEQ ID NO: 17 - DNA, 1410 bp, Arabidopsis thaliana
  G5287
tcaccatccatctttgttctttctcgtgcatggtgcaaccttctccatggccaaaaaatcacagctgcgtacccagaaaaacaatgttaccaccaatgacgataataatcttaacgtaaccaacactgtgaccaccaaggtgaaacgaacaaggagaagtgtccctagagactccccacctcaacgcagctcaatataccgaggagtcactaggcaccgatggacaggccgatacgaagctcatttgtgggacaaacattgctggaatgaatcacagaacaaaaaagggcgacaagtctaccttggcgcttatgacaatgaagaggcagcagcacatgcttatgatctagcagcactgaaatactggggtcaagataccattcttaattttccgttatcaaactacctgaacgaactgaaagaaatggagggtcaatcacgggaggagtatatcggatcgctgaggaggaaaagcagtggtttttctcggggaatttctaaatacagaggtgttgcaaggcatcatcataacggaaggtgggaggctcggattggcaaagtttttggcaataaatatctttacctcggaacttatgctacccaagaagaagctgctactgcctatgacctggcagccatagaataccgtggactcaatgctgtcaccaatttcgatctcagccgttacattaagtggcttaagcctaacaacaacaccaacaacgttatcgacgaccagattagtattaatctcactaacataaacaataataataattgcactaacagcttcaccccaagtcctgatcaagaacaagaagctagcttcttccacaacaaagattcactcaataatactattgtagaagaagtcacgttggtgccacatcagcctcgtccagcgagtgccacgtcagcattggagcttctacttcagtcatcaaagttcaaggaaatgatggagatgacatctgtggccaatctttcttcaacacagatggaatctgagttgccacagtgcacatttcctgatcacattcagacgtactttgagtatgaagattccaatagatatgaggaaggagatgatctcatgttcaagttcaacgagttcagctccattgtgccgttttaccaatgtgacgagttcgagagttgaagaagtcaggtttatataatgcatggaaaaaagaaactctgatatgtttgtttatttgtttaatttgttgattatgttaaagaccatattcataaatctttagctaattaaggtttaagtttttagaagagagatcatgtcattcacaactattataataagtggacttgttttcaatttgtgaacatgaaagtttattctttttatagcaacgtcgtcattaatcacataaaaatgaatattaatgcggc

SEQ ID NO: 18 - protein, 370 aa, Arabidopsis thaliana
  G5287 polypeptide, AP2 domains 49-118,151-212, linker domain 119-150
MAKKSQLRTQKNNVTTNDDNNLNVTNTVTTKVKRTRRSVPRDSPPQRSSIYRGVTRHRWTGRYEAHLWDKHCWNESQNKKGRQVYLGAYDNEEAAAHAYDLAALKYWGQDTILNFPLSNYLNELKEMEGQSREEYIGSLRRKSSGFSRGISKYRGVARHHHNGRWEARIGKVFGNKYLYLGTYATQEEAATAYDLAAIEYRGLNAVTNFDLSRYIKWLKPNNNTNNVIDDQISINLTNINNNNNCTNSFTPSPDQEQEASFFHNKDSLNNTIVEEVTLVPHQPRPASATSALELLLQSSKFKEMMEMTSVANLSSTQMESELPQCTFPDHIQTYFEYEDSNRYEEGDDLMFKFNEFSSIVPFYQCDEFES

SEQ ID NO: 19 - DNA, 1905 bp, Arabidopsis thaliana
  G15
atagaaagaagagaagcagaaaccaaaaaaagaaaccatgaagtctttttgtgataatgatgataataatcatagcaacacgactaatttgttagggttctcattgtcttcaaatatgatgaaaatgggaggtagaggaggtagagaagctatttactcatcttcaacttcttcagctgcaacttcttcttcttctgttccacctcaacttgttgttggtgacaacactagcaactttggtgtttgctatggatctaacccaaatggaggaatctattctcacatgtctgtgatgccactcagatctgatggttctctttgcttaatggaagctctcaacagatcttctcactcgaatcaccatcaagattcatctccaaaggtggaggatttctttgggacccatcacaacaacacaagtcacaaagaagccatggatcttagcttagatagtttattctacaacaccactcatgagcccaacacgactacaaactttcaagagttctttagcttccctcaaaccagaaaccatgaggaagaaactagaaattacgggaatgaccctagtttgacacatggagggtcttttaatgtaggggtatatggggaatttcaacagtcactgagcttatccatgagccctgggtcacaatctagctgcatcactggctctcaccaccaccaacaaaaccaaaaccaaaaccaccaaagccaaaaccaccagcagatctctgaagctcttgtggagacaagcgttgggtttgagacgacgacaatggcggctgcgaagaagaagaggggacaagaggatgttgtagttgttggtcagaaacagattgttcatagaaaatctatcgatacttttggacaacgaacttctcaataccgaggcgttacaagacatagatggactggtagatatgaagctcatctatgggacaatagtttcaagaaggaaggtcacagtagaaaaggaagacaagtttatctgggaggttatgatatggaggagaaagctgctcgagcatatgatcttgctgcactcaagtactggggtccctctactcacaccaatttctctgcggagaattatcagaaagagattgaagacatgaagaacatgactagacaagaatatgttgcacatttgagaaggaagagcagtggtttctctaggggtgcttccatctatagaggagtcacaagacatcaccagcatggaaggtggcaagcacggattggtagagtcgctggaaacaaagatctctaccttggaacttttggaacccaagaagaagctgcagaagcttacgatgtagcagcaattaagttccgtggcacaaatgctgtgactaactttgatatcacgaggtacgatgttgatcgtatcatgtctagtaacacactcttgtctggagagttagcgcgaaggaacaacaacagcattgtcgtcaggaatactgaagaccaaaccgctctaaatgctgttgtggaaggtggttccaacaaagaagtcagtactcccgagagactcttgagttttccggcgattttcgcgttgcctcaagttaatcaaaagatgttcggatcaaatatgggcggaaatatgagtccttggacatcaaaccctaatgctgagcttaagaccgtcgctcttactttgcctcagatgccggttttcgctgcttgggctgattcttgatcaacttcaatgactaactctggttttcttggtttagttgctaagtgttttggtttatctccggttttatccggtttgaactacaattcggtttagtttcgtcggtataaatagtatttgcttaggagcggtatatgtttcttttgagtagtattcatgtgaaacagaatgaatctctctataacatattattttaatgg

SEQ ID NO: 20 - protein, 555 aa, Arabidopsis thaliana
  G15 polypeptide, AP2 domains 282-351, 384-445, linker domain 352-383
MKSFCDNDDNNHSNTTNLLGFSLSSNMMKMGGRGGREAIYSSSTSSAATSSSSVPPQLVVGDNTSNFGVCYGSNPNGGIYSHMSVMPLRSDGSLCLMEALNRSSHSNHHQDSSPKVEDFFGTHHNNTSHKEAMDLSLDSLFYNTTHEPNTTTNFQEFFSFPQTRNHEEETRNYGNDPSLTHGGSFNVGVYGEFQQSLSLSMSPGSQSSCITGSHHHQQNQNQNHQSQNHQQISEALVETSVGFETTTMAAAKKKRGQEDVVVVGQKQIVHRKSIDTFGQRTSQYRGVTRHRWTGRYEAHLWDNSFKKEGHSRKGRQVYLGGYDMEEKAARAYDLAALKYWGPSTHTNFSAENYQKEIEDMKNMTRQEYVAHLRRKSSGFSRGASIYRGVTRHHQHGRWQARIGRVAGNKDLYLGTFGTQEEAAEAYDVAAIKFRGTNAVTNFDITRYDVDRIMSSNTLLSGELARRNNNSIVVRNTEDQTALNAVVEGGSNKEVSTPERLLSFPAIFALPQVNQKMFGSNMGGNMSPWTSNPNAELKTVALTLPQMPVFAAWADS

SEQ ID NO: 21 - protein, 70 aa, Arabidopsis thaliana
  G979 first AP2 domain
SIYRGVTRHRWTGRFEAHLWDKSSWNSIQNKKGKQVYLGAYDSEEAAAHTYDLAALKYWGPDTILNFPAE

SEQ ID NO: 22 - protein, 62 aa, Arabidopsis thaliana
  G979 second AP2 domain
SKYRGVARHHHNGRWEARIGRVFGNKYLYLGTYNTQEEAAAAYDMAAIEYRGANAVTNFDIS

SEQ ID NO: 23 - protein, 32 aa, Arabidopsis thaliana
  G979 linker sequence
TYTKELEEMQRVTKEEYLASLRRQSSGFSRGV

SEQ ID NO: 24 - protein, 71 aa, Zea mays
  G5297 first AP2 domain
SVYRGVTRHRWTGRFEAHLWDKHCLAALHNKKKGRQVYLGAYDSEEAAARAYDLAALKYWGPETLLNFPVE

SEQ ID NO: 25 - protein, 62 aa, Zea mays
  G5297 second AP2 domain
SKYRGVARHHHNGRWEARIGRVFGNKYLYLGTFDTQEEAAKAYDLAAIEYRGVNAVTNFDIS

SEQ ID NO: 26 - protein, 32 aa, Zea mays
  G5297 linker sequence
DYSSEMPEMEAVSREEYLASLRRRSSGFSRGV

SEQ ID NO: 27 - protein, 71 aa, Zea mays; identical to SEQ ID NO: 24
  G5286 first AP2 domain

SEQ ID NO: 28 - protein, 62 aa, Zea mays; identical to SEQ ID NO: 25
  G5286 second AP2 domain

SEQ ID NO: 29 - protein, 32 aa, Zea mays; identical to SEQ ID NO: 26
  G5286 linker sequence

SEQ ID NO: 30 - protein, 71 aa, Oryza sativa
  G5285 first AP2 domain
SIYRGVTRHRWTGRFEAHLWDKNCSTSLQNKKKGRQVYLGAYDSEEAAARAYDLAALKYWGPETVLNFPLE

SEQ ID NO: 31 - protein, 62 aa, Oryza sativa
  G5285 second AP2 domain
SKYRGVARHHHNGRWEARIGRVLGNKYLYLGTFDTQEEAAKAYDLAAIEYRGANAVTNFDIS

SEQ ID NO: 32 - protein, 32 aa, Oryza sativa
  G5285 linker sequence
EYEKERSEMEGVSREEYLASLRRRSSGFSRGV

SEQ ID NO: 33 - protein, 70 aa, Arabidopsis thaliana
  G5289 first AP2 domain
SIYRGVTRHRWTGRYEAHLWDKSSWNSIQNKKGKQVYLGAYDSEEAAAHTYDLAALKYWGPNTILNFPVE

SEQ ID NO: 34 - protein, 62 aa, Arabidopsis thaliana
  G5289 second AP2 domain
SKYRGVARHHHNGRWEARIGRVFGNKYLYLGTYNTQEEAAAAYDMAAIEYRGANAVTNFDIG

SEQ ID NO: 35 - protein, 32 aa, Arabidopsis thaliana
  G5289 linker sequence
TYTKELEEMQRCTKEEYLASLRRQSSGFSRGV

SEQ ID NO: 36 - protein, 70 aa, Arabidopsis thaliana
  G2131 first AP2 domain
SPYRGVTRHRWTGRYEAHLWDKNSWNDTQTKKGRQVYLGAYDEEEAAARAYDLAALKYWGRDTLLNFPLP

SEQ ID NO: 37 - protein, 62 aa, Arabidopsis thaliana
  G2131 second AP2 domain
SKYRGVARHHHNGRWEARIGRVFGNKYLYLGTYATQEEAAIAYDIAAIEYRGLNAVTNFDVS

SEQ ID NO: 38 - protein, 32 aa, Arabidopsis thaliana
  G2131 linker sequence
SYDEDVKEMEGQSKEEYIGSLRRKSSGFSRGV

SEQ ID NO: 39 - protein, 70 aa, Arabidopsis thaliana
  G2106 first AP2 domain
SVHRGVTRHRWTGRYEAHLWDKNSWNETQTKKGRQVYLGAYDEEDAAARAYDLAALKYWGRDTILNFPVN

SEQ ID NO: 40 - protein, 62 aa, Arabidopsis thaliana
  G2106 second AP2 domain
SKYRGVAKHHHNGRWEARIGRVFGNKYLYLGTYATQEEAAIAYDIAAIEYRGLNAVTNFDIS

SEQ ID NO: 41 - protein, 32 aa, Arabidopsis thaliana
  G2106 linker sequence
NYEEDIKEMESQSKEEYIGSLRRKSSGFSRGV

SEQ ID NO: 42 - protein, 70 aa, Arabidopsis thaliana
  G5288 first AP2 domain
SIYRGVTRHRWTGRYEAHLWDKHCWNESQNKKGRQVYLGAYDNEEAAAHAYDLAALKYWGQDTILNFPLS

SEQ ID NO: 43 - protein, 62 aa, Arabidopsis thaliana
  G5288 second AP2 domain
SKYRGVARHHHNGRWEARIGKVFGNKYLYLGTYATQEEAATAYDLAAIEYRGLNAVTNFDLS

SEQ ID NO: 44 - protein, 32 aa, Arabidopsis thaliana
  G5288 linker sequence
TYDEELKEMEGQSREEYIGSLRRKSSGFSRGV

SEQ ID NO: 45 - protein, 70 aa, Arabidopsis thaliana; identical to SEQ ID NO: 42
  G5287 first AP2 domain

SEQ ID NO: 46 - protein, 62 aa, Arabidopsis thaliana; identical to SEQ ID NO: 43
  G5287 second AP2 domain

SEQ ID NO: 47 - protein, 32 aa, Arabidopsis thaliana
  G5287 linker sequence
NYLNELKEMEGQSREEYIGSLRRKSSGFSRGI

SEQ ID NO: 48 - protein, 70 aa, Arabidopsis thaliana
  G15 first AP2 domain
SQYRGVTRHRWTGRYEAHLWDNSFKKEGHSRKGRQVYLGGYDMEEKAARAYDLAALKYWGPSTHTNFSAE

SEQ ID NO: 49 - protein, 62 aa, Arabidopsis thaliana
  G15 second AP2 domain
SIYRGVTRHHQHGRWQARIGRVAGNKDLYLGTFGTQEEAAEAYDVAAIKFRGTNAVTNFDIT

SEQ ID NO: 50 - protein, 32 aa, Arabidopsis thaliana
  G15 linker sequence
NYQKEIEDMKNMTRQEYVAHLRRKSSGFSRGA

SEQ ID NO: 51 - protein, 71 aa, Arabidopsis thaliana
  misc_feature (2)..(2): Xaa can be Ile, Val or Leu
SXYRGVTRHRWTGRXEAHLWDKXXXXXXXNKKXGXQVYLGAYDSEEAAAXXYDLAALKYWGPXTXLNFPXE

SEQ ID NO: 52 - protein, 71 aa, Arabidopsis thaliana
  misc_feature (2)..(3): Xaa can be any naturally occurring amino acid
SXXRGVTRHRWTGRXEAHLWDKXXXXXXXXKKXGXQVYLGAYDXEXAAAXXYDLAALKYWGXXTXLNFPXX

SEQ ID NO: 53 - protein, 62 aa, Arabidopsis thaliana
  misc_feature (23)..(23): Xaa can be any naturally occurring amino acid
SKYRGVARHHHNGRWEARIGRVXGNKYLYLGTXXTQEEAAXAYDXAAIEYRGXNAVTNFDIX

SEQ ID NO: 54 - protein, 62 aa, Arabidopsis thaliana
  misc_feature (8)..(8): Xaa can be Arg or Lys
SKYRGVAXHHHNGRWEARIGXVXGNKYLYLGTXXTQEEAAXAYDXAAIEYRGXNAVTNFDXX

SEQ ID NO: 55 - protein, 31 aa, Arabidopsis thaliana
  misc_feature (1)..(1): Xaa can be any naturally occurring amino acid
XYXXEXXEMXXXXXEEYLASLRRXSSGFSRG

SEQ ID NO: 56 - protein, 31 aa, Arabidopsis thaliana
  misc_feature (1)..(1): Xaa can be any naturally occurring amino acid
XYXXXXXEMXXXXXEEYXXSLRRXSSGFSRG

(19) United States
(12) Patent Application Publication
(10) Pub. No.: US 2009/0192305 A1
(43) Pub. Date: Jul. 30, 2009

(54) AP2 TRANSCRIPTION FACTORS FOR MODIFYING PLANT TRAITS

(75) Inventors: Jose Luis Riechmann, Barcelona (ES); Oliver Ratcliffe, Oakland, CA (US); T. Lynne Reuber, San Mateo, CA (US); Robert A. Creelman, Castro Valley, CA (US); Luc J. Adam, Hayward, CA (US); Roderick W. Kumimoto, Norman, OK (US)

Correspondence Address: Mendel Biotechnology, Inc., 3935 Point Eden Way, Hayward, CA, 94545, US

(73) Assignee: Mendel Biotechnology, Inc., Hayward, CA (US)

(21) Appl. No.: 12/356,991

(22) Filed: Jan. 21, 2009

Related U.S. Application Data
(63) Continuation-in-part of application No. 11/986,992, filed on Nov. 26, 2007.
Application No. 11/986,992 is a division of application No. 10/412,699, filed on Apr. 10, 2003, now Pat. No. 7,345,217.
Application No. 10/412,699 is a continuation-in-part of application No. 10/295,403, filed on Nov. 15, 2002, now abandoned.
Application No. 10/295,403 is a division of application No. 09/394,519, filed on Sep. 13, 1999, now abandoned.
(60) Provisional application No. 60/113,409, filed on Dec. 22, 1998.

(51) Int. Cl. C07H 21/00

(52) U.S. Cl. 536/23.6

(57) ABSTRACT

This invention relates to polynucleotide and polypeptide transcription factor sequences that are of use for the transformation of plants. The AP2 transcription factors include G979, polynucleotide and polypeptide SEQ ID NOs: 1 and 2, respectively, and phylogenetically-related sequences.

### CROSS REFERENCE TO RELATED APPLICATIONS

This application is a continuation-in-part application of U.S. application Ser. No. 11/986,992, filed Nov. 26, 2007 (pending), which is a divisional application of U.S. application Ser. No. 10/412,699, filed Apr. 10, 2003 (now issued as U.S. Pat. No. 7,345,217), which is a continuation-in-part application of U.S. application Ser. No. 10/295,403, filed Nov. 15, 2002 (abandoned), which is a divisional application of U.S. application Ser. No. 09/394,519, filed Sep. 13, 1999 (abandoned), which claims the benefit under 35 U.S.C. § 119(e) to U.S. Provisional application No. 60/113,409, filed Dec. 22, 1998. The disclosure of each patent or patent application of this paragraph is hereby incorporated by reference herein in its entirety.

### FIELD OF THE INVENTION

The present invention relates to nucleic acids encoding transcription factors and their use in plant improvement.

### BACKGROUND OF THE INVENTION

The G979 polynucleotide sequence, SEQ ID NO: 1, was first identified in a BAC-end sequence B25031, which comprises a partial G979 sequence. The G979 polynucleotide corresponds to gene T12E 18_{—}20 (BAC T12E 18, AL132971). No information was available about the function(s) of G979 in these citations.

### SUMMARY OF THE INVENTION

This invention pertains to the polynucleotide and polypeptide sequences of the AP2 transcription factor G979, SEQ ID NOs: 1 and 2, respectively, and phylogenetically-related sequences. The invention also pertains to a nucleic acid construct, a host cell transformed with and comprising said nucleic acid construct, or a plant transformed with and comprising said nucleic acid construct, wherein the nucleic acid construct comprises a regulatory sequence and SEQ ID NO: 1 or a sequence that is phylogenetically-related to SEQ ID NO: 1.

### BRIEF DESCRIPTION OF THE SEQUENCE LISTING AND DRAWINGS

The Sequence Listing provides exemplary polynucleotide and polypeptide sequences of the invention. The traits associated with the use of the sequences are included in the Examples.

Incorporation of the Sequence Listing. The copy of the Sequence Listing, being submitted electronically with this patent application, provided under 37 CFR § 1.821-1.825, is a read-only memory computer-readable file in ASCII text format. The Sequence Listing is named “MBI-0087CIP_ST25.txt”, the electronic file of the Sequence Listing was created on Jan. 9, 2009, and is 81 kilobytes in size (measured in MS-WINDOWS). The Sequence Listing is herein incorporated by reference in its entirety.

FIG. 1 shows a phylogenetic tree of G979 and closely-related related full length proteins that was constructed using Accelrys© Gene v 2.5 software. The parameters used for building the tree were:

Tree building method: UPGMA

Distance: uncorrected (“p”)

Bootstrap no. of replications: 1000

The arrow pointing to node “A” represents a common ancestral sequence from which the G979 subclade, containing sequences most closely related to G979, was derived. Similarly, the arrow pointing to node “B” represents a common ancestral sequence from which the greater G979 clade derived, and contains somewhat less closely related sequences. Data obtained with two G979 clade sequences in a C/N sensing assay confirmed the conservation of both function and structure within the larger G979 clade (data presented below).

### DETAILED DESCRIPTION OF THE INVENTION

The present invention relates to polynucleotides and polypeptides. Throughout this disclosure, various information sources are referred to and/or are specifically incorporated. The information sources include scientific journal articles, patent documents, textbooks, and World Wide Web browser-inactive page addresses. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the invention.

As used herein and in the appended claims, the singular forms “a”, “an”, and “the” include the plural reference unless the context clearly dictates otherwise. Thus, for example, a reference to “a host cell” includes a plurality of such host cells, and a reference to “a stress” is a reference to one or more stresses and equivalents thereof known to those skilled in the art, and so forth.

### DEFINITIONS

“Polynucleotide” is a nucleic acid molecule comprising a plurality of polymerized nucleotides, for example, at least about 15 consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single-stranded or double-stranded DNA or RNA. The polynucleotide optionally comprises modified bases or a modified backbone. The polynucleotide can be, for example, genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, primer, oligomer, element, target, and probe and is preferably single-stranded.

A “recombinant polynucleotide” is a polynucleotide that is not in its native state, for example, the polynucleotide comprises a nucleotide sequence not found in nature, or the polynucleotide is in a context other than that in which it is naturally found, for example, separated from nucleotide sequences with which it typically is in proximity in nature, or adjacent (or contiguous with) nucleotide sequences with which it typically is not in proximity. For example, the sequence at issue can be cloned into a nucleic acid construct, or otherwise recombined with one or more additional nucleic acid.

An “isolated polynucleotide” is a polynucleotide, whether naturally occurring or recombinant, that is present outside the cell in which it is typically found in nature, whether purified or not. Optionally, an isolated polynucleotide is subject to one or more enrichment or purification procedures, for example, cell lysis, extraction, centrifugation, precipitation, or the like.

“Gene” or “gene sequence” refers to the partial or complete coding sequence of a gene, its complement, and its 5′ or 3′ untranslated regions. A gene is also a functional unit of inheritance, and in physical terms is a particular segment or sequence of nucleotides along a molecule of DNA (or RNA, in the case of RNA viruses) involved in producing a polypeptide chain. The latter may be subjected to subsequent processing such as chemical modification or folding to obtain a functional protein or polypeptide. A gene may be isolated, partially isolated, or found with an organism's genome.

Operationally, genes may be defined by the cis-trans test, a genetic test that determines whether two mutations occur in the same gene and that may be used to determine the limits of the genetically active unit (Rieger et al. (1976) Glossary of Genetics and Cytogenetics: Classical and Molecular, 4th ed., Springer Verlag, Berlin). A gene generally includes regions preceding (“leaders”; upstream) and following (“trailers”; downstream) the coding region. A gene may also include intervening, non-coding sequences, referred to as “introns”, located between individual coding segments, referred to as “exons”. Most genes have an associated promoter region, a regulatory sequence 5′ of the transcription initiation codon (there are some genes that do not have an identifiable promoter). The function of a gene may also be regulated by enhancers, operators, and other regulatory elements.

A “polypeptide” is an amino acid sequence comprising a plurality of consecutive polymerized amino acid residues for example, at least about 15 consecutive polymerized amino acid residues. The polypeptide optionally comprises modified amino acid residues, naturally occurring amino acid residues not encoded by a codon, non-naturally occurring amino acid residues.

“Protein” refers to an amino acid sequence, oligopeptide, peptide, polypeptide or portions thereof whether naturally occurring or synthetic.

A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, for example, more than about 5% enriched, more than about 10% enriched, or more than about 20%, or more than about 50%, or more, enriched, that is, alternatively denoted: 105%, 110%, 120%, 150% or more, enriched relative to wild type standardized at 100%. Such an enrichment is not the result of a natural response of a wild-type plant. Alternatively, or additionally, the isolated polypeptide is separated from other cellular components with which it is typically associated, for example, by any of the various protein purification methods herein.

The invention also encompasses production of DNA sequences that encode polypeptides and derivatives, or fragments thereof, entirely by synthetic chemistry. After production, the synthetic sequence may be inserted into any of the many available nucleic acid constructs and cell systems using reagents well known in the art. Moreover, synthetic chemistry may be used to introduce mutations into a sequence encoding polypeptides or any fragment thereof.

The term “plant” includes whole plants, shoot vegetative organs/structures (for example, leaves, stems, rhizomes, and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like), calli, protoplasts, and cells (for example, guard cells, egg cells, and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, multicellular algae, and unicellular algae.

A “control plant” as used in the present invention refers to a plant cell, seed, plant component, plant tissue, plant organ or whole plant used to compare against transformed, transgenic or genetically modified plant for the purpose of identifying an enhanced phenotype in the transformed, transgenic or genetically modified plant. A control plant may in some cases be a transformed or transgenic plant line that comprises an empty nucleic acid construct or marker gene, but does not contain the recombinant polynucleotide of the present invention that is expressed in the transformed, transgenic or genetically modified plant being evaluated. In general, a control plant is a plant of the same line or variety as the transformed, transgenic or genetically modified plant being tested. A suitable control plant would include a genetically unaltered or non-transgenic plant of the parental line used to generate a transformed or transgenic plant herein.

“Wild type” or “wild-type”, as used herein, refers to a plant cell, seed, plant component, plant tissue, plant organ or whole plant that has not been genetically modified or treated in an experimental sense. Wild-type cells, seed, components, tissue, organs or whole plants may be used as controls to compare levels of expression and the extent and nature of trait modification with cells, tissue or plants of the same species in which a polypeptide's expression is altered, for example, in that it has been knocked out, overexpressed, or ectopically expressed.

“Transformation” refers to the transfer of a foreign polynucleotide sequence into the genome of a host organism such as that of a plant or plant cell, or introduction of a foreign polynucleotide sequence into plant or plant cell such that is expressed and results in production of protein. Typically, the foreign genetic material has been introduced into the plant by human manipulation, but any method can be used as one of skill in the art recognizes. Examples of methods of plant transformation include Agrobacterium-mediated transformation (De Blaere et. al. (1987) Meth. Enzymol., vol. 153: 277-292) and biolistic methodology (U.S. Pat. No. 4,945,050 to Klein et al.).

A “transformed plant”, which may also be referred to as a “transgenic plant” or “transformant”, generally refers to a plant, a plant cell, plant tissue, seed or calli that has been through, or is derived from a plant cell that has been through, a stable or transient transformation process in which a “nucleic acid construct” that contains at least one exogenous polynucleotide sequence is introduced into the plant. The “nucleic acid construct” contains genetic material that is not found in a wild-type plant of the same species, variety or cultivar, or may contain extra copies of a native sequence under the control of its native promoter. In some embodiments the a nucleic acid sequence transformed into a plant may be derived from the host plant, but by its incorporation into a nucleic acid construct, represents an element not found in a wild-type plant of the same species, variety or cultivar.

An “untransformed plant” is a plant that has not been through the transformation process.

A “nucleic acid construct” may comprise a polypeptide-encoding sequence operably linked (that is, under regulatory control of) to appropriate inducible, cell-specific, tissue-specific, cell-enhanced, tissue-enhanced, condition-enhanced, developmental, or constitutive regulatory sequences that allow for the controlled expression of polypeptide. The expression vector or cassette can be introduced into a plant by transformation or by breeding after transformation of a parent plant. A plant refers to a whole plant as well as to a plant part, such as seed, fruit, leaf, or root, plant tissue, plant cells or any other plant material, for example, a plant explant, to produce a recombinant plant (for example, a recombinant plant cell comprising the nucleic acid construct) as well as to progeny thereof, and to in vitro systems that mimic biochemical or cellular components or processes in a cell.

“Cell-enhanced” and “tissue-enhanced” regulation refer to the control of gene or protein expression, for example, by a promoter, which drives expression that is not necessarily totally restricted to a single type of cell or tissue, but where expression is elevated in particular cells or tissues to a greater extent than in other cells or tissues within the organism.

A “condition-enhanced” promoter refers to a promoter that activates a gene in response to a particular environmental stimulus, for example, an abiotic stress, infection caused by a pathogen, light treatment, etc., and that drives expression in a unique pattern which may include expression in specific cell and/or tissue types within the organism (as opposed to a constitutive expression pattern that occurs in all cell types of an organism at all times).

### DESCRIPTION OF THE SPECIFIC EMBODIMENTS

The data presented herein represent the results obtained in experiments with polynucleotides that may be transformed into plants for the purpose of enhancing various plant traits.

### G979-Related Transcription Factor Polynucleotide and Polypeptide Sequences

Background Information.

The G979 polynucleotide sequence, SEQ ID NO: 1, was first identified in a BAC-end sequence B25031, which comprises a partial G979 sequence. The G979 polynucleotide corresponds to gene T12E18_{—}20 (Arabidopsis thaliana DNA chromosome 3, BAC clone T12E18, Nov. 12, 1999). No information was available about the function(s) of G979 in these citations.

Discoveries Related to the G979 Sequences

The complete sequence of G979, SEQ ID NO: 1 was obtained using a “Rapid Amplification of cDNA Ends” (RACE) method to obtain the full length sequence from the RNA transcript. RACE is used to produce cDNA copies of an RNA sequence of interest by a reverse transcription step followed by PCR amplification of the resulting cDNA copies. The amplified cDNA copies are then sequenced and assembled to obtain a full length sequence. The encoded protein, SEQ ID NO: 2, is a member of the AP2 subfamily of transcription factors and contains two AP2 domains.

The function of G979, SEQ ID NO: 1, was studied using both transgenic plants in which G979 was expressed under the control of the Cauliflower mosaic virus 35S promoter, and also with a knockout (KO) line with a T-DNA insertion in the gene. The T-DNA insertion of the KO line lay in an intron, located in between the exons coding for the second AP2 domain of the protein (at position 1544 bp downstream of the first base of the start codon in the genomic sequence), and was thus expected to result in a strong or null mutation. Whereas constitutive expression of G979 produced deleterious effects, the analysis of G979 KO mutant plants proved informative about the function of the gene. Seeds homozygous for the T-DNA insertion within the G979 polynucleotide showed delayed ripening, slow germination, and developed into small, poorly fertile plants, suggesting that G979 might be involved in seed development processes.

The difficulty in initially isolating, from heterozygous plants, progeny that were homozygous for the T-DNA insertion raised the possibility that homozygosity for that allele was lethal or conditionally lethal. Siliques of heterozygous plants were examined for seed abnormalities. In accordance with a Mendelian segregation for a mongenic trait, approximately 25% of the seeds contained in young green siliques were pale in coloration. In older, brown siliques, approximately 25% of the seeds were green and appeared slow ripening, whereas the remaining seeds were brown. Thus, it seemed likely that the seeds with altered development were homozygous for the T-DNA insertion, whereas the normal seeds were wild type and heterozygous segregants.

Furthermore, it was observed that approximately 25% of the seed from G979 KO heterozygous plants showed impaired (delayed) germination. Upon germination, these seeds produced extremely tiny seedlings that often did not survive transplantation. A few homozygous plants, small and sickly looking, could be grown, and produced siliques that contained seeds that were small and wrinkled compared to wild type.

A second, different, T-DNA insertion allele for G979 was identified as part of a TAIL PCR screen. This insertion is at position 2242 downstream of the first base of the start codon in the genomic sequence, within an intron, and should result in the truncation of approximately 50% of the coding sequence, thus producing a strong or null mutation. Progeny of the heterozygous plant carrying that T-DNA insertion was either wild-type or heterozygous for the mutation, providing additional evidence for the disruption of G979 being the cause of the phenotypic alterations detected.

The mutant phenotypes displayed by plants carrying these two independent alleles provided strong genetic evidence that the G979 protein has a critical function in controlling normal seed development and maturation.

An initial analysis of 35S::G979 transformants revealed that the overexpressors were generally smaller than wild type and developed spindly inflorescences which sometimes carried abnormal flowers, with compromised fertility. G979 (SEQ ID NO: 2) overexpressors also exhibited altered carbon-nitrogen (C/N) sensing, being more tolerant to low nitrogen conditions than control plants. This observation suggests that G979 functions to regulate carbon and nitrogen flux within the plant. Overexpression of another clade member sequence, G2131 (SEQ ID NO: 12), also produced plants with increased tolerance to low nitrogen conditions in a C/N sensing screen. 35S::G2131 transformants were further shown to have increased campesterol in leaves, indicating that the transcription factor regulates the production or accumulation of organic molecules of this class.

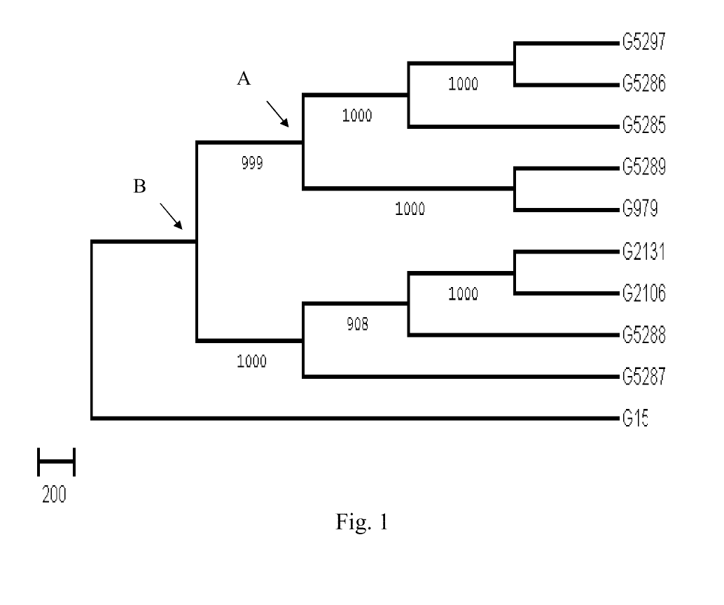

Table 1 provides a list of G979 subclade sequences (derived from ancestral node “A” in FIG. 1) and broader clade sequences (derived from ancestral node “B” in FIG. 1), and identifies the species from which these sequences are derived (Column 2), the SEQ ID NO. of each of the polypeptides (Column 3), the percentage identity to the G979 sequence (Column 4), and the amino acids (counting from the N-terminus of each polypeptide), SEQ ID NOs., and the percentage identity to G979 of the first and second AP2 domains in Columns 5-10. Note that the “first” and “second” AP2 domains are comprised with G979 clade polypeptide sequences as counted from the N-terminus.

TABLE 1
G979 subclade and clade sequences and identification of AP2 domains

| | | | | | | | | | |
|---|---|---|---|---|---|---|---|---|---|
| | Col. 2 | | Col. 4 | Col. 5 | Col. 6 | Col. 7 | Col. 8 | Col. 9 | Col. 10 |
| | Plant species | | % | 1st AP2 | 1st AP2 | % identity of | 2nd AP2 | 2nd AP2 | % identity of |
| | from which | Col. 3 | identity | domain | domain | 1st AP2 domain | domain | domain | 2nd AP2 domain |
| Col. 1 | GID is | SEQ ID | of GID | amino acid | SEQ ID | to 1st AP2 domain | amino acid | SEQ ID | to 2nd AP2 domain |
| GID | derived* | NO: | to G979 | coordinates | NO: | of G979 | coordinates | NO: | of G979 |

G979 subclade sequences

| | | | | | | | | | |
|---|---|---|---|---|---|---|---|---|---|
| G979 | At | 2 | 100% | 64-133 | 21 | 100% | 166-227 | 22 | 100% |
| G5297 | Zm | 4 | 49.0% | 63-133 | 24 | 78.8% | 166-227 | 25 | 91.9% |
| G5286 | Zm | 6 | 48.8% | 66-136 | 27 | 78.8% | 169-230 | 28 | 91.9% |
| G5285 | Os | 8 | 46.3% | 79-149 | 30 | 83.0% | 182-243 | 31 | 91.9% |
| G5289 | Bn | 10 | 84.2% | 61-130 | 33 | 95.7% | 163-224 | 34 | 98.3% |

G979 clade sequences outside of the G979 subclade

| | | | | | | | | | |
|---|---|---|---|---|---|---|---|---|---|
| G2131 | At | 12 | 49.0% | 51-120 | 36 | 80.0% | 153-214 | 37 | 91.9% |
| G2106 | At | 14 | 45.5% | 57-126 | 39 | 78.5% | 166-227 | 40 | 91.9% |
| G5288 | Os | 16 | 40.2% | 54-123 | 42 | 78.5% | 156-217 | 43 | 88.7% |
| G5287 | Gm | 18 | 42.1% | 49-118 | 45 | 84.2% | 151-212 | 46 | 90.3% |

Related sequence outside the G979 domain

| | | | | | | | | | |
|---|---|---|---|---|---|---|---|---|---|
| G15 | At | 20 | 41.3% | 282-351 | 48 | 70.0% | 384-445 | 49 | 75.8% |

Table 2 provides a list of G979 subclade sequences and lade sequences and identifies the species from which these sequences are derived (Column 2), the SEQ ID NO. of a linker subsequence between the AP2 domains of each of the polypeptides (Column 3), and the amino acids (counting from the N-terminus of each polypeptide) and the percentage identity to the similar linker sequence of G979 (Columns 4 and 5).

TABLE 2
G979 subclade and clade sequences and identification of linker
sequences between first and second AP2 domains

| | | | | |
|---|---|---|---|---|
| | | | | Col. 5 |
| | Col. 2 | Col. 3 | Col. 4 | % identity |
| | Plant species | Linker | Linker | of linker |
| Col. 1 | from which | SEQ ID | amino acid | to linker |
| GID | GID is derived* | NO: | coordinates | of G979 |

G979 subclade sequences

| | | | | |
|---|---|---|---|---|
| G979 | At | 23 | 134-165 | 100% |
| G5297 | Zm | 26 | 134-165 | 68.7% |
| G5286 | Zm | 29 | 137-168 | 68.7% |
| G5285 | Os | 32 | 150-181 | 71.8% |
| G5289 | Bn | 35 | 131-162 | 96.8% |

G979 clade sequences outside of the G979 subclade

| | | | | |
|---|---|---|---|---|
| G2131 | At | 38 | 121-152 | 59.3% |
| G2106 | At | 41 | 134-165 | 59.3% |
| G5288 | Os | 44 | 124-155 | 65.6% |
| G5287 | Gm | 47 | 119-150 | 59.3% |

Related sequence outside the G979 domain

| | | | | |
|---|---|---|---|---|
| G15 | At | 50 | 352-383 | 59.3% |

*Abbreviations for Tables 1 and 2: At (Arabidopsis thaliana), Bn (Brassica napus), Gm (Glycine max), Os (Oryza saliva), and Zm (Zea mays)

Thus, the sequences that have thus far been found to be within the G979 clade include those with similar evolutionarily-conserved functions and a first AP2 domain with at least 79%, or at least 80%, or at least 83%, or at least 84%, or at least 96%, or about 100% to the first AP2 domain of G979, SEQ ID NO: 21.

The sequences that have thus far been found to be within the G979 clade with similar evolutionarily-conserved functions include those with a second AP2 domain with at least 88%, or at least 90%, or at least 91%, or at least 98%, or about 100% to the second AP2 domain of G979, SEQ ID NO: 22.

The sequences that have thus far been found to be within the G979 clade with similar evolutionarily-conserved functions include those with a linker domain located between the first and second AP2 domains with at least 59%, or at least 65%, or at least 68%, or at least 71%, or at least 96%, or about 100% to the similar linker domain of G979, SEQ ID NO: 23.

The sequences that have thus far been found to be within the G979 subclade possess a consensus first AP2 domain comprising SEQ ID NO: 51:

SX₁YRGVTRHRWTGRX₂EAHLWDKXXXXX₃X₄XNKKXGX₅QVYLGAYDSE
EAAAXXYDLAALKYWGPXTX₆LNFPXE

where X is any naturally occurring amino acid, except:

### X₁ can be Ile, Val or Leu;

### X₂ can be Phe or Tyr;

### X₃ can be Ser or Ala;

### X₄ can be Ile, Val or Leu;

### X₅ can be Arg or Lys; and

### X₆ can be Ile, Val or Leu.

The sequences that have thus far been found to be within the broader G979 clade possess a consensus first AP2 domain comprising SEQ ID NO: 52:

SXXRGVTRHRWTGRX₁EAHLWDKXXXXXXXXKKXGX₂QVYLGAYDXEX₃A
AAXXYDLAALKYWGXXTX₄LNFPXX

where X is any naturally occurring amino acid, except:

### X₁ can be Tyr or Phe;

### X₂ can be Arg or Lys;

### X₃ can be Glu or Asp; and

### X₄ can be Ile, Val or Leu.

The sequences that have thus far been found to be within the G979 subclade possess a consensus linker domain comprising SEQ ID NO: 55:

XYXXEXXEMX₁XXX₂X₃EEYLASLRRX₄SSGFSRG

where X is any naturally occurring amino acid, except:

### X₁ can be Glu or Gln;

### X₂ can be Ser or Thr;

### X₃ can be Arg or Lys; and

### X₄ can be Lys, Arg or Gln.

The sequences that have thus far been found to be within the broader G979 clade possess a consensus linker domain comprising SEQ ID NO: 56:

XYXXX₁XXEMX₂XXX₃X₄EEYX₅XSLRRX₆SSGFSRG

### X₁ can be Glu or Asp;

### X₂ can be Glu or Gln;

### X₃ can be Ser or Thr;

### X₄ can be Arg or Lys;

### X₅ can be Ile, Leu or Val; and

### X₆ can be Lys, Arg or Gln.

The sequences that have thus far been found to be within the G979 subclade possess a consensus second AP2 domain comprising SEQ ID NO: 53:

SKYRGVARHHHNGRWEARIGRVXGNKYLYLGTX₁X₂TQEEAAXAYDX₃AAIEYRGXNAVTNFDIX₄

where X is any naturally occurring amino acid, except:

### X₁ can be Tyr or Phe;

### X₂ can be Asp or Asn;

### X₃ can be Met or Leu; and

### X₄ can be Ser or Gly.

The sequences that have thus far been found to be within the broader G979 clade possess a consensus second AP2 domain comprising SEQ ID NO: 54:

SKYRGVAX₁HHHNGRWEARIGX₂VXGNKYLYLGTX₃XTQEEAAXAYDXAA
IEYRGXNAVTNFDX₄X₅

where X is any naturally occurring amino acid, except:

### X₁ can be Arg or Lys;

### X₂ can be Arg or Lys;

### X₃ can be Tyr or Phe;

### X₄ can be Ile, Leu or Val; and

### X₅ can be Ser or Gly.

### Sequence Variations

It will readily be appreciated by those of skill in the art that the instant invention includes any of a variety of polynucleotide sequences provided in the Sequence Listing or capable of encoding polypeptides that function similarly to those provided in the Sequence Listing. Due to the degeneracy of the genetic code, many different polynucleotides can encode identical and/or substantially similar polypeptides in addition to those sequences illustrated in the Sequence Listing. Nucleic acids having a sequence that differs from the sequences shown in the Sequence Listing, or complementary sequences, that encode functionally equivalent peptides (that is, peptides having some degree of equivalent or similar biological activity) but differ in sequence from the sequence shown in the sequence listing due to degeneracy in the genetic code, are also within the scope of the invention.

Altered polynucleotide sequences encoding polypeptides include those sequences with deletions, insertions, or substitutions of different nucleotides, resulting in a polynucleotide encoding a polypeptide with at least one functional characteristic of the instant polypeptides. Included within this definition are polymorphisms which may or may not be readily detectable using a particular oligonucleotide probe of the polynucleotide encoding the instant polypeptides, and improper or unexpected hybridization to allelic variants, with a locus other than the normal chromosomal locus for the polynucleotide sequence encoding the instant polypeptides.

Sequence alterations that do not change the amino acid sequence encoded by the polynucleotide are termed “silent” variations. With the exception of the codons ATG and TGG, encoding methionine and tryptophan, respectively, any of the possible codons for the same amino acid can be substituted by a variety of techniques, for example, site-directed mutagenesis, available in the art. Accordingly, any and all such variations of a sequence selected from the above table are a feature of the invention.

In addition to silent variations, other conservative variations that alter one, or a few amino acids in the encoded polypeptide, can be made without altering the function of the polypeptide. For example, substitutions, deletions and insertions introduced into the sequences provided in the Sequence Listing are also envisioned. Such sequence modifications can be engineered into a sequence by site-directed mutagenesis (for example, Olson et al., Smith et al., Zhao et al., and other articles in Wu (ed.) Meth. Enzymol. (1993) vol. 217, Academic Press) or the other methods known in the art or noted herein. Amino acid substitutions are typically of single residues; insertions usually will be on the order of about from 1 to 10 amino acid residues; and deletions will range about from 1 to 30 residues. In preferred embodiments, deletions or insertions are made in adjacent pairs, for example, a deletion of two residues or insertion of two residues. Substitutions, deletions, insertions or any combination thereof can be combined to arrive at a sequence. The mutations that are made in the polynucleotide encoding the transcription factor should not place the sequence out of reading frame and should not create complementary regions that could produce secondary mRNA structure. Preferably, the polypeptide encoded by the DNA performs the desired function.

Conservative substitutions are those in which at least one residue in the amino acid sequence has been removed and a different residue inserted in its place. Such substitutions generally are made in accordance with the Table 3 when it is desired to maintain the activity of the protein. Table 3 shows amino acids which can be substituted for an amino acid in a protein and which are typically regarded as conservative substitutions.

TABLE 3
Possible conservative amino acid substitutions

| Amino Acid | |
|---|---|
| Residue | Conservative substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

The polypeptides provided in the Sequence Listing have a novel activity, such as, for example, regulatory activity. Although all conservative amino acid substitutions (for example, one basic amino acid substituted for another basic amino acid) in a polypeptide will not necessarily result in the polypeptide retaining its activity, it is expected that many of these conservative mutations would result in the polypeptide retaining its activity. Most mutations, conservative or non-conservative, made to a protein but outside of a conserved domain required for function and protein activity will not affect the activity of the protein to any great extent.

### Identifying Polynucleotides or Polypeptides Related to the Disclosed Sequences by Percent Identity

With the aid of a computer, one of skill in the art could identify all of the polypeptides, or all of the nucleic acids that encode a polypeptide, with, for example, at least 85% identity to the sequences provided herein and in the Sequence Listing. Electronic analysis of sequences may be conducted with a software program such as the MEGALIGN program (DNASTAR, Inc. Madison, Wis.). The MEGALIGN program can create alignments between two or more sequences according to different methods, for example, the clustal method (see, for example, Higgins and Sharp (1988) Gene 73: 237-244). The clustal algorithm groups sequences into clusters by examining the distances between all pairs. The clusters are aligned pairwise and then in groups. Other alignment algorithms or programs may be used, including FASTA, BLAST, or ENTREZ, FASTA and BLAST, and which may be used to calculate percent similarity. These are available as a part of the GCG sequence analysis package (University of Wisconsin, Madison, Wis.), and can be used with or without default settings. ENTREZ is available through the National Center for Biotechnology Information. In one embodiment, the percent identity of two sequences can be determined by the GCG program with a gap weight of 1, for example, each amino acid gap is weighted as if it were a single amino acid or nucleotide mismatch between the two sequences (see U.S. Pat. No. 6,262,333).

Software for performing BLAST analyses is publicly available, for example, through the National Center for Biotechnology Information (see internet website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) J. Mol. Biol. 215: 403-410, Altschul (1993) J. Mol. Evol. 36: 290-300). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff (1989) Proc. Natl. Acad. Sci. USA 89: 10915). Unless otherwise indicated for comparisons of predicted polynucleotides, “sequence identity” refers to the % sequence identity generated from a tblastx using the NCBI version of the algorithm at the default settings using gapped alignments with the filter “off” (see, for example, internet website at www.ncbi.nlm.nih.gov/).

Other techniques for alignment are described by Doolittle, ed. (1996) Methods in Enzymology, vol. 266: “Computer Methods for Macromolecular Sequence Analysis” Academic Press, Inc., San Diego, Calif., USA. Preferably, an alignment program that permits gaps in the sequence is utilized to align the sequences. The Smith-Waterman is one type of algorithm that permits gaps in sequence alignments (see Shpaer (1997) Methods Mol. Biol. 70: 173-187). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Percent identity can also be determined manually, by comparing the entire length of a sequence of sequence with another in an optimal alignment.

Generally, the percentage similarity between two polypeptide sequences, for example, sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between polynucleotide sequences can also be counted or calculated by other methods known in the art, for example, the Jotun Hein method (see, for example, Hein (1990) Methods Enzymol. 183: 626-645) Identity between sequences can also be determined by other methods known in the art, for example, by varying hybridization conditions (see US Patent Application No. US20010010913).

At the polynucleotide level, the sequences described herein in the Sequence Listing, and the sequences of the invention by virtue of a paralogous or homologous relationship with the sequences described in the Sequence Listing, will typically share at least 30%, or 40% nucleotide sequence identity, preferably at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to one or more of the listed full-length sequences, or to a region of a listed sequence excluding or outside of the region(s) encoding a known consensus sequence or consensus DNA-binding site, or outside of the region(s) encoding one or all conserved domains. The degeneracy of the genetic code enables major variations in the nucleotide sequence of a polynucleotide while maintaining the amino acid sequence of the encoded protein.

At the polypeptide level, the sequences described herein in the Sequence Listing and Tables 1 and 2, and the sequences of the invention by virtue of a paralogous, orthologous, or homologous relationship with the sequences described in the Sequence Listing or in Table 1 or Table 2, including full-length sequences and conserved domains, will typically share at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% amino acid sequence identity or more sequence identity to one or more of the listed full-length sequences, or to a listed sequence but excluding or outside of the known consensus sequence or consensus DNA-binding site.

### Identifying Polynucleotides Related to the Disclosed Sequences by Hybridization

Polynucleotides homologous to the sequences illustrated in the Sequence Listing and tables can be identified, for example, by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof), as described in more detail in the references cited below (for example, Sambrook et al. (1989) Molecular Cloning: A Laboratory Manual, 2nd Ed., Cold Spring Harbor Laboratory, Cold Spring Harbor, N.Y.; Schroeder et al. (2002) Current Biol. 12, 1462-1472; Berger and Kimmel (1987), “Guide to Molecular Cloning Techniques”, in Methods in Enzymology, vol. 152, Academic Press, Inc., San Diego, Calif.; and Anderson and Young (1985) “Quantitative Filter Hybridisation”, In: Hames and Higgins, ed., Nucleic Acid Hybridisation A Practical Approach. Oxford, IRL Press, 73-111).

Encompassed by the invention are polynucleotide sequences that are capable of hybridizing to the claimed polynucleotide sequences, including any of the polynucleotides within the Sequence Listing, and fragments thereof under various conditions of stringency (see, for example, Wahl and Berger (1987) Methods Enzymol. 152: 399-407; and Kimmel (1987) Methods Enzymol. 152: 507-511). In addition to the nucleotide sequences listed in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

With regard to hybridization, conditions that are highly stringent, and means for achieving them, are well known in the art. See, for example, Sambrook et al., 1989; Berger, 1987, pages 467-469; and Anderson and Young, 1985, all supra.

Stability of DNA duplexes is affected by such factors as base composition, length, and degree of base pair mismatch. Hybridization conditions may be adjusted to allow DNAs of different sequence relatedness to hybridize. The melting temperature (Tₘ) is defined as the temperature when 50% of the duplex molecules have dissociated into their constituent single strands. The melting temperature of a perfectly matched duplex, where the hybridization buffer contains formamide as a denaturing agent, may be estimated by the following equations:

(I) DNA-DNA:

Tₘ(° C.)=81.5+16.6(log [Na+])+0.41(% G+C)−0.62(% formamide)−500/L

(II) DNA-RNA:

Tₘ(° C.)=79.8+18.5(log [Na+])+0.58(% G+C)+0.12(% G+C)²−0.5(% formamide)−820/L

(III) RNA-RNA:

Tₘ(° C.)=79.8+18.5(log [Na+])+0.58(% G+C)+0.12(% G+C)²−0.35(% formamide)-820/L

where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson and Young, 1985, supra). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tₘ−5° C. to Tₘ−20° C., moderate stringency at Tₘ−20° C. to Tₘ₋₃₅° C. and low stringency at Tₘ−35° C. to Tₘ−50° C. for duplex >150 base pairs. Hybridization may be performed at low to moderate stringency (25-50° C. below Tₘ), followed by post-hybridization washes at increasing stringencies. Maximum rates of hybridization in solution are determined empirically to occur at Tₘ−25° C. for DNA-DNA duplex and Tₘ−15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or Northern blot for hybridization of complementary nucleic acids that have more than 100 complementary residues is about 5° C. to 20° C. lower than the thermal melting point (Tₘ) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. More preferably, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, for example, to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, for example, formamide, whereas high stringency hybridization may be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. with formamide present. Varying additional parameters, such as hybridization time, the concentration of detergent, for example, sodium dodecyl sulfate (SDS) and ionic strength, are well known to those skilled in the art. Various levels of stringency are accomplished by combining these various conditions as needed.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps will preferably be less than about 30 mM NaCl and 3 mM trisodium citrate, and most preferably less than about 15 mM NaCl and 1.5 mM trisodium citrate.

Thus, hybridization and wash conditions that may be used to bind and remove polynucleotides with less than the desired homology to the nucleic acid sequences or their complements that encode the present polypeptides include, for example:

6×SSC and 1% SDS at 65° C.;

50% formamide, 4×SSC at 42° C.; or

0.5×SSC to 2.0×SSC, 0.1% SDS at 50° C. to 65° C.;

with a first wash step of, for example, 10 minutes at about 42° C. with about 20% (v/v) formamide in 0.1×SSC, and with, for example, a subsequent wash step with 0.2×SSC and 0.1% SDS at 65° C. for 10, 20 or 30 minutes. An example of an amino acid sequence of the invention would include one encoded by a polynucleotide selected from the Sequence Listing and nucleic acid sequence fragments encoding various proteins that have been or can be used for cloning and nucleic acid sequence fragments that encode various functional (e.g., regulatory or indicator) polypeptides, and which can be incorporated into nucleic acid constructs for cloning purposes.

Useful variations on these conditions will be readily apparent to those skilled in the art.

A person of skill in the art would not expect substantial variation among polynucleotide species encompassed within the scope of the present invention because the highly stringent conditions set forth in the above formulae yield structurally similar polynucleotides.

If desired, one may employ wash steps of even greater stringency, including about 0.2×SSC, 0.1% SDS at 65° C. and washing twice, each wash step being about 30 minutes, or about 0.1×SSC, 0.1% SDS at 65° C. and washing twice for 30 minutes. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homologs, wash steps may be performed at a lower temperature, for example, 50° C.

An example of a low stringency wash step employs a solution and conditions of at least 25° C. in 30 mM NaCl, 3 mM trisodium citrate, and 0.1% SDS over 30 minutes. Greater stringency may be obtained at 42° C. in 15 mM NaCl, with 1.5 mM trisodium citrate, and 0.1% SDS over 30 minutes. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art (see, for example, US Patent Application No. US20010010913).

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid encoding a polypeptide known as of the filing date of the application. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, for example, a fluorescent label, a colorimetric label, a radioactive label, or the like. Labeled hybridization or PCR probes for detecting related polynucleotide sequences may be produced by oligolabeling, nick translation, end-labeling, or PCR amplification using a labeled nucleotide.

Encompassed by the invention are polynucleotide sequences that are capable of hybridizing to the claimed polynucleotide sequences, including any of the polynucleotides within the Sequence Listing, and fragments thereof under various conditions of stringency (see, for example, Wahl and Berger, 1987, pages 399-407; and Kimmel, 1987). In addition to the nucleotide sequences in the Sequence Listing, full length cDNA, orthologs, and paralogs of the present nucleotide sequences may be identified and isolated using well-known methods. The cDNA libraries, orthologs, and paralogs of the present nucleotide sequences may be screened using hybridization methods to determine their utility as hybridization target or amplification probes.

### EXAMPLES

It is to be understood that this invention is not limited to the particular devices, machines, materials and methods described. Although particular embodiments are described, equivalent embodiments may be used to practice the invention.

The invention, now being generally described, will be more readily understood by reference to the following examples, which are included merely for purposes of illustration of certain aspects and embodiments of the present invention and are not intended to limit the invention. It will be recognized by one of skill in the art that a polypeptide that is associated with a particular first trait may also be associated with at least one other, unrelated and inherent second trait which was not predicted by the first trait.

### Example I

### Project Types, Constructs and Cloning Information

Constructs were used to modulate the activity of sequences of the invention. An individual project was defined as the analysis of lines for a particular construct (for example, this might include G979 lines that constitutively overexpressed a sequence of the invention). Generally, a full-length wild-type version of a gene was directly fused to a promoter that drove its expression in transformed or transgenic plants. Such a promoter could be a constitutive promoter such as the CaMV 35S promoter, or the native promoter of that gene. Alternatively, a promoter that drives tissue-enhanced, tissue-specific, or conditional expression could be used in similar studies.

Expression of a given polynucleotide from a particular promoter was achieved by a direct-promoter fusion construct in which that sequence was cloned directly behind the promoter of interest. A direct fusion approach has the advantage of allowing for simple genetic analysis if a given promoter-polynucleotide line is to be crossed into different genetic backgrounds at a later date.

As an alternative to direct promoter fusion, a two-component expression system may be used to drive transcription factor expression. For the two-component system, two separate constructs are used: Promoter::LexA-GAL4TA and opLexA::TF. The first of these (Promoter::LexA-GAL4TA) comprises a desired promoter cloned in front of a LexA DNA binding domain fused to a GAL4 activation domain. The construct vector backbone also carries a selectable marker (such as kanamycin resistance), and optionally, also an opLexA::GFP cassette or other suitable reporter (the latter allows the monitoring of expression patterns produced by the promoter included in the construct). It should be noted that a transcription factor may be expressed from any of a wide range of different promoters using a two component method. Transgenic lines are obtained containing the first component, and a line is selected that shows reproducible expression of the reporter gene in the desired pattern through a number of generations. A population, which typically is homozygous, is established for that line, and the population is supertransformed with the second construct (opLexA::TF) carrying the transcription factor sequence of interest cloned behind a LexA operator site. This second construct vector backbone also contains a selectable marker, e.g., sulfonamide resistance. The two-component approach might also be implemented by a genetic crossing strategy as an alternative to supertransformation.

Each of the above methods offers a number of pros and cons. A direct fusion approach allows for much simpler genetic analysis if a given promoter-transcription factor line was to be crossed into different genetic backgrounds at a later date. The two-component method, on the other hand, potentially allows for stronger expression to be obtained via an amplification of transcription, and could be also be a means to ensure that a trait is only expressed in F1 hybrid seed that are produced from crossing two parental lines each of which carries only one of the two transgene components.

### Example II

### Transformation of Agrobacterium with the Expression Vector

After the expression constructs are generated, the constructs are used to transform Agrobacterium tumefaciens cells expressing the gene products. The stock of Agrobacterium tumefaciens cells for transformation is made as described by Nagel et al. (1990) FEMS Microbiol Letts. 67: 325-328. Agrobacterium strain ABI is grown in 250 ml LB medium (Sigma) overnight at 28° C. with shaking until an absorbance over 1 cm at 600 nm (A₆₀₀) of 0.5-1.0 is reached. Cells are harvested by centrifugation at 4,000×g for 15 min at 4° C. Cells are then resuspended in 250 μl chilled buffer (1 mM HEPES, pH adjusted to 7.0 with KOH). Cells are centrifuged again as described above and resuspended in 125 μl chilled buffer. Cells are then centrifuged and resuspended two more times in the same HEPES buffer as described above at a volume of 100 μl and 750 μl, respectively. Resuspended cells are then distributed into 40 μl aliquots, quickly frozen in liquid nitrogen, and stored at −80° C.

Agrobacterium cells are transformed with constructs prepared as described above following the protocol described by Nagel et al. (supra). For each DNA construct to be transformed, 50-100 ng DNA (generally resuspended in 10 mM Tris-HCl, 1 mM EDTA, pH 8.0) is mixed with 40 μl of Agrobacterium cells. The DNA/cell mixture is then transferred to a chilled cuvette with a 2 mm electrode gap and subject to a 2.5 kV charge dissipated at 25 μF and 200 μF using a Gene Pulser II apparatus (Bio-Rad, Hercules, Calif.). After electroporation, cells are immediately resuspended in 1.0 ml LB and allowed to recover without antibiotic selection for 2-4 hours at 28° C. in a shaking incubator. After recovery, cells are plated onto selective medium of LB broth containing 100 μg/ml spectinomycin (Sigma) and incubated for 24-48 hours at 28° C. Single colonies are then picked and inoculated in fresh medium. The presence of the plasmid construct is verified by PCR amplification and sequence analysis.

### Example III

### Transformation of Plants with Agrobacterium tumefaciens

After transformation of Agrobacterium tumefaciens with the constructs or plasmid vectors containing the gene of interest, single Agrobacterium colonies are identified, propagated, and used to transform plants. In the example here, transformation of Arabidopsis plants is disclosed, but the constructs could be introduced into any plant species, including crops such as corn, soybean, cotton, rice, canola, Crambe, Miscanthus, sugarcane, rutabaga, and tomato, which is amenable to transformation and using transformation methodologies which have been optimized for those species. Briefly, 500 ml cultures of LB medium containing 50 mg/l kanamycin are inoculated with the colonies and grown at 28° C. with shaking for 2 days until an optical absorbance at 600 nm wavelength over 1 cm (A₆₀₀) of >2.0 is reached. Cells are then harvested by centrifugation at 4,000×g for 10 min, and resuspended in infiltration medium (½×Murashige and Skoog salts (Sigma), 1× Gamborg's B-5 vitamins (Sigma), 5.0% (w/v) sucrose (Sigma), 0.044 μM benzylamino purine (Sigma), 200 μl/l Silwet L-77 (Lehle Seeds) until an A₆₀₀ of 0.8 is reached.

Prior to transformation, Arabidopsis thaliana seeds (ecotype Columbia) are sown at a density of ˜10 plants per 4″ pot onto Pro-Mix BX potting medium (Hummert International) covered with fiberglass mesh (18 mm×16 mm). Plants are grown under continuous illumination (50-75 μE/m²/sec) at 22-23° C. with 65-70% relative humidity. After about 4 weeks, primary inflorescence stems (bolts) are cut off to encourage growth of multiple secondary bolts. After flowering of the mature secondary bolts, plants are prepared for transformation by removal of all siliques and opened flowers.

The pots are then immersed upside down in the mixture of Agrobacterium infiltration medium as described above for 30 sec, and placed on their sides to allow draining into a 1′×2′ flat surface covered with plastic wrap. After 24 h, the plastic wrap is removed and pots are turned upright. The immersion procedure is repeated one week later, for a total of two immersions per pot. Seeds are then collected from each transformation pot and analyzed following the protocol described below. Other standard methods of plant transformation, such as particle bombardment, or tissue culture-based Agrobacterium cocultivation could also be applied to transform Arabidopsis, or any other plant species of interest.

### Example IV

### Identification of Arabidopsis Primary Transformants

Seeds collected from the transformation pots are sterilized essentially as follows. Seeds are dispersed into in a solution containing 0.1% (v/v) Triton X-100 (Sigma) and sterile water and washed by shaking the suspension for 20 min. The wash solution is then drained and replaced with fresh wash solution to wash the seeds for 20 min with shaking. After removal of the ethanol/detergent solution, a solution containing 0.1% (v/v) Triton X-100 and 30% (v/v) bleach (CLOROX; Clorox Corp. Oakland Calif.) is added to the seeds, and the suspension is shaken for 10 min. After removal of the bleach/detergent solution, seeds are then washed five times in sterile distilled water. The seeds are stored in the last wash water at 4° C. for 2 days in the dark before being plated onto antibiotic selection medium (1× Murashige and Skoog salts (pH adjusted to 5.7 with 1M KOH), 1× Gamborg's B-5 vitamins, 0.9% phytagar (Life Technologies), and 50 mg/l kanamycin). Seeds are germinated under continuous illumination (50-75 μE/m²/sec) at 22-23° C. After 7-10 days of growth under these conditions, kanamycin resistant primary transformants (T1 generation) are visible and obtained. At this stage, transformed plants are subjected to detailed microscopic analysis to verify that each cloned promoter fragment is driving gene expression in the desired cell type-specific pattern. While still growing on primary selection plates, seedlings are placed under a fluorescent dissecting microscope so that the opLexA::GFP protein pattern can be verified (if applicable). This pattern, since it is controlled via a GAL4-LexA 2-component system, should also represent the pattern of the TF of interest. Plants showing a correct SUC2 promoter pattern, for example, show high levels of fluorescence in the vascular tissue of the leaves and roots. Plants containing the correct RBCS1A promoter pattern show strong expression in green tissue, but not in roots, and plants comprising a seed promoter should later show expression in developing seeds. Seedlings are then transplanted to soil (Pro-Mix BX potting medium) for continued growth and characterization at subsequent developmental stages.

Primary transformants are self fertilized and progeny seeds (T₂) collected; seedlings carrying the transgene are selected (using either the selectable marker or via molecular approaches) and analyzed. The expression levels of the recombinant polynucleotides in the transformants typically varies from about a 5% expression level increase to at least a 100% expression level increase, in tissue samples from the transgenic lines compared to those from wild-type controls, in the target tissue(s) where the transcription factor is being expressed. Similar observations are made with respect to polypeptide level expression.

### Example V

### Morphological and Physiological Analyses

### Morphological Analyses

Morphological analyses were performed to determine whether changes in polypeptide levels affect plant growth and development. This was primarily carried out on the T1 generation, when at least 10-20 independent lines were examined. However, in cases where a phenotype required confirmation or detailed characterization, plants from subsequent generations were also analyzed.

Primary transformants were selected on MS medium with 0.3% sucrose and 50 mg/l kanamycin. T2 and later generation plants were selected in the same manner, except that kanamycin was used at 35 mg/l. In cases where lines carry a sulfonamide marker (as in all lines generated by super-transformation), Transformed seeds were selected on MS medium with 0.3% sucrose and 1.5 mg/l sulfonamide. KO lines were usually germinated on plates without a selection. Seeds were cold-treated (stratified) on plates for three days in the dark (in order to increase germination efficiency) prior to transfer to growth cabinets. Initially, plates were incubated at 22° C. under a light intensity of approximately 100 microEinsteins for 7 days. At this stage, transformants were green, possessed the first two true leaves, and were easily distinguished from bleached kanamycin or sulfonamide-susceptible seedlings. Resistant seedlings were then transferred onto soil (Sunshine® potting mix, Sun Gro Horticulture®, Bellevue, Wash.). Following transfer to soil, trays of seedlings were covered with plastic lids for 2-3 days to maintain humidity while they became established. Plants were grown on soil under fluorescent light at an intensity of 70-95 microEinsteins and a temperature of 18-23° C. Light conditions consisted of a 24-hour photoperiod unless otherwise stated. In instances where alterations in flowering time were apparent, flowering time was re-examined under both 12-hour and 24-hour light to assess whether the phenotype was photoperiod dependent. Under our 24-hour light growth conditions, the typical generation time (seed to seed) was approximately 14 weeks.

Because many aspects of Arabidopsis development are dependent on localized environmental conditions, plants were evaluated in comparison to controls in the same flat. Controls for transformed lines were generally wild-type plants or transformed plants harboring an empty transformation vector selected on kanamycin or sulfonamide. Careful examination was made at the following stages: seedling (1 week), rosette (2-3 weeks), flowering (4-7 weeks), and late seed set (8-12 weeks). Seed was also inspected. Seedling morphology was assessed on selection plates. At all other stages, plants were macroscopically evaluated while growing on soil. All significant differences (including alterations in growth rate, size, leaf and flower morphology, coloration, and flowering time) were recorded, but routine measurements were not taken if no differences were apparent.

### Altered C/N Sensing

Transgenic plants overexpressing a G979 subclade sequence (G979, SEQ ID NO: 2) or a G979 clade sequence (G2131, SEQ ID NO: 12) were subjected to C/N sensing studies and showed positive results. These assays were intended to find genes that allowed more plant growth upon deprivation of nitrogen, or which modulate plant metabolism to adjust to changes in sugar levels and regulate carbon flux into different types of organic molecules within the plant. Indeed, recent data of Lam et al. (Plant Physiology 2003, vol. 132: 926-935) showed that a C/N assay could be used identify genes that produce improvements in seed nutrient content. Nitrogen is a major nutrient affecting plant growth and development that ultimately impacts yield and stress tolerance. The C/N assays monitored growth and the appearance of stress symptons such as anthocyanins or media with high sugar levels or which is nitrogen deficient. In all higher plants, inorganic nitrogen is first assimilated into glutamate, glutamine, aspartate and asparagine, the four amino acids used to transport assimilated nitrogen from sources (e.g. leaves) to sinks (e.g. developing seeds). This process is regulated by light, as well as by C/N metabolic status of the plant. A C/N sensing assay was thus used to look for alterations in the mechanisms plants use to sense internal levels of carbon and nitrogen metabolites which could activate signal transduction cascades that regulate the transcription of nitrogen-assimilatory genes. To determine whether these mechanisms are altered, we exploited the observation that wild-type plants grown on media containing high levels of sucrose (3%) without a nitrogen source accumulate high levels of anthocyanins. This sucrose induced anthocyanin accumulation can be relieved by the addition of either inorganic or organic nitrogen. For these N additions we used glutamine (1 mM) as a nitrogen source since it also serves as a compound used to transport nitrogen in plants. A positive result was obtained when seedlings of the transgenic overexpression line showed visibly more vigor and/or lower levels of stress-induced compounds (such as anthocyanins) in a C/N assay, relative to controls which lacked the transgene.

Germination assays to determine altered C/N sensing were performed in aseptic conditions. Growing the plants under controlled temperature and humidity on sterile medium produces uniform plant material that has not been exposed to additional stresses (such as water stress) which could cause variability in the results obtained. Where possible, assay conditions were originally tested in a blind experiment with controls that had phenotypes related to the conditions tested.

Prior to plating, seed for all experiments were surface sterilized in the following manner: (1) 5 minute incubation with mixing in 70% ethanol, (2) 20 minute incubation with mixing in 30% bleach, 0.01% triton-X 100, (3) 5× rinses with sterile water, (4) Seeds were re-suspended in 0.1% sterile agarose and stratified at 4° C. for 3-4 days.

All germination assays follow modifications of the same basic protocol. Sterile seeds were sown on the conditional media that has a basal composition of 80% MS+Vitamins. Plates were incubated at 22° C. under 24-hour light (120-130 μm⁻² s⁻¹) in a growth chamber. Evaluation of germination and seedling vigor was generally performed five days after planting.

### Example VI

### Characteristics of Transgenic Plants that Overexpress G979 Clade Member

Arabidopsis thaliana plant lines overexpressing G979 (SEQ ID NO: 2) demonstrated altered carbon-nitrogen (C/N) sensing, being more tolerant to low nitrogen conditions than control plants. Overexpression of another clade member sequence, G2131 (SEQ ID NO: 12), also produced Arabidopsis plants with increased tolerance to low nitrogen conditions in a C/N sensing screen. 35S::G2131 transformants were also shown, through GC-FID analysis, to have increased campesterol in leaves.

All references, publications, patent documents, web pages, and other documents cited or mentioned herein are hereby incorporated by reference in their entirety for all purposes. Although the invention has been described with reference to specific embodiments and examples, it should be understood that one of ordinary skill can make various modifications without departing from the spirit of the invention. The scope of the invention is not limited to the specific embodiments and examples provided.

What is claimed is:

1. An isolated polynucleotide sequence encoding a polypeptide comprising, in order from N-terminus to C-terminus, SEQ ID NO: 52, SEQ ID NO: 56 and SEQ ID NO: 54, wherein expression of the polypeptide in a plant confers altered carbon-nitrogen balance sensing, increased tolerance to low nitrogen conditions, reduced size, or reduced fertility, as compared to a control plant.

2. The isolated polynucleotide sequence of claim 1, wherein the polypeptide comprises SEQ ID NO: 2.

3. The isolated polynucleotide sequence of claim 1, wherein the isolated polynucleotide comprises SEQ ID NO: 1.

4. An isolated polynucleotide sequence encoding a polypeptide comprising a first AP2 domain having at least 80% identity to amino acids 64-133 of SEQ ID NO: 2, a linker domain having at least 59% identity to amino acids 134-165 of SEQ ID NO: 2, and a second AP2 domain having at least 91% identity to amino acids 166-227 of SEQ ID NO: 2, wherein expression of the polypeptide in a plant confers altered carbon-nitrogen balance sensing or increased tolerance to low nitrogen conditions.

5. The isolated polynucleotide sequence of claim 4, wherein the first AP2 domain has at least 95% identity to amino acids 64-133 of SEQ ID NO: 2, the linker domain has at least 71% identity to amino acids 134-165 of SEQ ID NO: 2, and the second AP2 domain has at least 91% identity to amino acids 166-227 of SEQ ID NO: 2.

6. The isolated polynucleotide sequence of claim 4, wherein the first AP2 domain has at least 95% identity to amino acids 64-133 of SEQ ID NO: 2, the linker domain has at least 96% identity to amino acids 134-165 of SEQ ID NO: 2, and the second AP2 domain has at least 91% identity to amino acids 166-227 of SEQ ID NO: 2.

7. An isolated polynucleotide sequence encoding SEQ ID NO: 2.
